(19) United States
(12) Patent Application Publication
(10) Pub. No.: US 2016/0367826 A1
(43) Pub. Date: Dec. 22, 2016

(54) SYSTEM AND METHOD FOR DETERMINING APPROPRIATE STEERING TABLES FOR DISTRIBUTING STIMULATION ENERGY AMONG MULTIPLE NEUROSTIMULATION ELECTRODES

(71) Applicant: Boston Scientific Neuromodulation Corporation, Valencia, CA (US)

(72) Inventors: Sridhar Kothandaraman, Valencia, CA (US); Carla Mann Woods, Beverly Hills, CA (US); Kerry Bradley, Glendale, CA (US); James R. Thacker, Eureka, MO (US); Sivakumar Karnati, Canoga Park, CA (US)

(21) Appl. No.: 15/252,772

(22) Filed: Aug. 31, 2016

Related U.S. Application Data
(63) Continuation of application No. 12/614,942, filed on Nov. 9, 2009, now abandoned.
(60) Provisional application No. 61/113,973, filed on Nov. 12, 2008.

(51) Int. Cl. A61N 1/372; A61N 1/36

(52) U.S. Cl. CPC A61N 1/37247; A61N 1/36071; A61N 1/37264; A61N 1/36157

(57) ABSTRACT

A method, computer medium, and system for programming a control device are provided. The control device is configured for controlling electrical stimulation energy provided to a plurality of electrode leads that are physically implanted within a patient in a side-by-side lead configuration. Electrical energy is conveying from the electrode leads to create a stimulation region within the patient. The stimulation region is automatically shifted along the electrode leads (e.g., by selecting and using at least one navigation table) in accordance with an electrical current shifting pattern that is based on a stagger of the side-by-side lead configuration. At least one stimulation parameter set is selected based on the effectiveness of the shifted stimulation region, and the control device is programmed with the selected stimulation parameter set(s).

### RELATED APPLICATION

The present application is a continuation of U.S. application Ser. No. 12/614,942, filed Nov. 9, 2009, which claims the benefit under 35 U.S.C. §119 to U.S. provisional patent application Ser. No. 61/113,973, filed Nov. 12, 2008. The foregoing application is hereby incorporated by reference into the present application in its entirety.

### FIELD OF THE INVENTION

The present invention relates to tissue stimulation systems, and more particularly, to a system and method for programming an implantable tissue stimulator.

### BACKGROUND OF THE INVENTION

Spinal cord stimulation (SCS) is a well-accepted clinical method for reducing pain in certain populations of patients. Spinal cord stimulator and other implantable tissue stimulator systems come in two general types: radio-frequency (RF)-controlled and fully implanted. The type commonly referred to as an “RF” system includes an external RF transmitter inductively coupled via an electromagnetic link to an implanted receiver-stimulator connected to one or more leads with one or more electrodes for stimulating tissue. The power source, e.g., a battery, for powering the implanted receiver, as well as control circuitry to command the receiver-stimulator, is contained in the RF transmitter—a hand-held sized device typically worn on the patient's belt or carried in a pocket. Data/power signals are transcutaneously coupled from a cable-connected transmission coil connected to the RF transmitter and placed over the implanted receiver-stimulator. The implanted receiver-stimulator receives the signal and generates the stimulation. In contrast, the fully implanted type of stimulating system contains the control circuitry, as well as a power supply, e.g., a battery, all within an implantable pulse generator (IPG), so that once programmed and turned on, the IPG can operate independently of external hardware. The IPG is turned on and off and programmed to generate the desired stimulation pulses from an external portable programming device using transcutaneous electromagnetic or RF links.

In both the RF-controlled or fully implanted systems, the electrode leads are implanted along the dura of the spinal cord. Individual wires within one or more electrode leads connect with each electrode on the lead. The electrode leads exit the spinal column and attach to one or more electrode lead extensions, when necessary. The electrode leads or extensions are typically tunneled along the torso of the patient to a subcutaneous pocket where the receiver-stimulator or IPG is implanted. The RF transmitter or IPG can then be operated to generate electrical pulses that are delivered, through the electrodes, to the targeted tissue, and in particular, the dorsal column and dorsal root fibers within the spinal cord. The stimulation creates the sensation known as paresthesia, which can be characterized as an alternative sensation that replaces the pain signals sensed by the patient. Individual electrode contacts (the “electrodes”) are arranged in a desired pattern and spacing in order to create an electrode array.

The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied in SCS include the amplitude, width, and rate of the electrical pulses provided through the electrode array. Each electrode combination, along with the electrical pulse parameters, can be referred to as a “stimulation parameter set.”

Amplitude may be measured in milliamps, volts, etc., as appropriate, depending on whether the system provides stimulation from current sources or voltage sources. With some SCS systems, and in particular, SCS systems with independently controlled current or voltage sources, the distribution of the current to the electrodes (including the case of the receiver-stimulator or IPG, which may act as an electrode) may be varied such that the current is supplied via numerous different electrode configuration. In different configurations, the electrodes may provide current (or voltage) in different relative percentages of positive and negative current (or voltage) to create different fractionalized electrode configurations.

As briefly discussed above, an external control device, such as an RF controller or portable programming device, can be used to instruct the receiver-stimulator or IPG to generate electrical stimulation pulses in accordance with the selected stimulation parameters. Typically, the stimulation parameters programmed into the external device, itself, can be adjusted by manipulating controls on the external device itself to modify the electrical stimulation provided by the SCS system to the patient. However, the number of electrodes available, combined with the ability to generate a variety of complex stimulation pulses, presents a huge selection of stimulation parameter sets to the clinician or patient.

To facilitate such selection, the clinician generally programs the external control device, and if applicable the IPG, through a computerized programming system. This programming system can be a self-contained hardware/software system, or can be defined predominantly by software running on a standard personal computer (PC). The PC or custom hardware may actively control the characteristics of the electrical stimulation generated by the receiver-stimulator or IPG to allow the optimum stimulation parameters to be determined based on patient feedback and to subsequently program the RF transmitter or portable programming device with the optimum stimulation parameters. The computerized programming system may be operated by a clinician attending the patient in several scenarios.

For example, in order to achieve an effective result from SCS, the lead or leads must be placed in a location, such that the electrical stimulation will cause paresthesia. The paresthesia induced by the stimulation and perceived by the patient should be located in approximately the same place in the patient's body as the pain that is the target of treatment. If a lead is not correctly positioned, it is possible that the patient will receive little or no benefit from an implanted SCS system, since the lead location will strongly determine the paresthesia location(s) on the patient's body. Thus, correct lead placement can mean the difference between effective and ineffective pain therapy. When electrical leads are implanted within the patient, the computerized programming system, in the context of an operating room (OR) mapping procedure, may be used to instruct the RF transmitter or IPG to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the IPG, with a set of stimulation parameters that best addresses the painful site. Thus, the navigation session may be used to pinpoint the stimulation region or areas correlating to the pain. Such programming ability is particularly advantageous after implantation should the leads gradually or unexpectedly move, thereby relocating the paresthesia away from the pain site. By reprogramming the external control device, the stimulation region can often be moved back to the effective pain site without having to reoperate on the patient in order to reposition the lead and its electrode array.

One known computerized programming system for SCS is called the Bionic Navigator®, available from Boston Scientific Neuromodulation, Sylmar, Calif. The Bionic Navigator® is a software package that operates on a suitable PC and allows clinicians to program stimulation parameters into an external handheld programmer (referred to as a remote control). Each set of stimulation parameters, including fractionalized current distribution to the electrodes (as percentage cathodic current, percentage anodic current, or off), programmed by the Bionic Navigator® may be stored in both the Bionic Navigator® and the remote control and combined into a stimulation program that can then be used to stimulate multiple regions within the patient.

Prior to creating the stimulation programs, the Bionic Navigator® may be operated by a clinician in a “manual mode” to manually select the percentage cathodic current and percentage anodic current flowing through the electrodes, or may be operated by the clinician in a “navigation mode” to electrically “steer” the current along the implanted leads in real-time, thereby allowing the clinician to determine the most efficacious stimulation parameter sets that can then be stored and eventually combined into stimulation programs. In the navigation mode, the Bionic Navigator® can store selected fractionalized electrode configurations that can be displayed to the clinician as marks representing corresponding stimulation regions relative to the electrode array.

The Bionic Navigator® performs current steering in accordance with a steering or navigation table. For example, as shown in Appendix A, an exemplary navigation table, which includes a series of reference electrode combinations (for a lead of 8 electrodes) with associated fractionalized current values (i.e., fractionalized electrode configurations), can be used to gradually steer electrical current from one basic electrode combination to the next, thereby electronically steering the stimulation region along the leads. The marks can then be created from selected fractionalized electrode configurations within the navigation table that can be combined with the electrical pulse parameters to create one or more stimulation programs.

For example, the navigation table can be used to gradually steer current between a basic electrode combination consisting of a cathodic electrode 3 and an anodic electrode 5 (represented by stimulation set 161) and either a basic electrode combination consisting of a cathodic electrode 3 and an anodic electrode 1 (represented by stimulation set 141) or a basic electrode combination consisting of a cathodic electrode 3 and an anodic electrode 6 (represented by stimulation set 181). That is, electrical current can be incrementally shifted from anodic electrode 5 to the anodic electrode 1 as one steps upward through the navigation table from stimulation set 161 to stimulation set 141, and from anodic electrode 5 to anodic electrode 6 as one steps downward through the navigation table from stimulation set 161 to stimulation set 181. The step size of the current should be small enough so that steering of the current does not result in discomfort to the patient, but should be large enough to allow refinement of a basic electrode combination in a reasonable amount of time.

Current SCS systems use one or more navigation tables that are designed for a specific lead configuration, so that the focus of the stimulation energy is gradually shifted between electrodes of the leads whose physical configuration corresponds to the designed lead configuration.

For example, a navigation table may be constructed for a side-by-side lead configuration, so that a single focus of the stimulation energy can be gradually shifted up, down, left and right within the electrodes when the leads are physically placed in a side-by-side configuration.

As another example, a navigation table may be constructed for an in-line lead configuration (e.g., one in the cervical region to treat a peripheral neuropathy in the right arm, and the other in the lower thoracic region to treat lower back pain), so that two foci of the stimulation energy can be independently shifted up and down the respective leads. This lead configuration would require a navigation table that does not result in the sharing of current between the electrodes on the respective leads.

Notably, a navigation table that was specifically designed to provide current steering for a side-by-side lead configuration that would result in the sharing of current between the electrodes of the respective leads could not be effectively used to steer current in an in-line lead configuration designed to separately treat different pain regions—else the navigation table would result in confusing, possibly simultaneous stimulation. Likewise, a navigation table that was specifically designed to provide current steering for an in-line lead configuration that would result in no sharing of current between the electrodes of the respective leads could not be effectively used to steer current in a side-by-side lead configuration.

Thus, it should be appreciated that the choice of navigation tables is critical to the smoothness and focus of the stimulation energy provided by the electrodes. If these navigation tables are not appropriately chosen, then the stimulation patterns may be haphazard, and thereby may not optimize the paresthesia provided to the patient, and may even frustrate the patient and the physician/clinician to the point where steering is not clinically used. To provide a smooth transition of the focus of the stimulation energy for each pain region to be treated, the Bionic Navigator®, based on input from the physician/clinician, automatically selects the navigation table that corresponds to the actual configuration in which the leads are implanted within the patient.

With respect to side-by-side electrode configurations, although current navigation tables assume that the electrode leads are not staggered, the electrode leads may, in fact, have a stagger (i.e., the degree to which the first electrode of one lead is vertically offset from the first electrode of another lead) either because the physician initially implanted the electrode leads in the manner to maximize the therapeutic effect of the stimulation or because the electrode leads subsequently migrated from an initially unstaggered configuration. If a navigation table that was designed to steer current between the electrodes of an unstaggered side-by-side lead configuration were to be used to steer current between the electrodes of a staggered side-by-side lead configuration, it is possible that at least one cathode of one lead would be adjacent an anode of the other lead, thereby possibly resulting in ineffective stimulation of the patient.

There, thus, remains a need for an improved method and system for programming multiple electrical stimulation leads that have been physically implanted in a side-by-side configuration.

### SUMMARY OF THE INVENTION

In accordance with a first aspect of the present inventions, a method of programming a control device is provided. The control device is configured for controlling electrical stimulation energy provided to a plurality of electrode leads that are physically implanted within a patient (e.g., adjacent a spinal cord of the patient) in a side-by-side lead configuration. The control device may be, e.g., an implantable pulse generator, an external trial stimulator, or an external device configured for controlling the electrical stimulation energy output by the implantable device to the electrode leads.

The method comprises selecting one of a plurality of different lead stagger configurations. As examples, the plurality of different lead stagger configurations may comprise a non-staggered lead configuration and a staggered lead configuration or the plurality of different lead stagger configurations may comprise differently staggered lead configurations. The method further comprises selecting at least one navigation table that corresponds to the selected lead configuration from a plurality of different navigation tables, with each of the navigation tables including a series of stimulation parameter sets. In one method, the stimulation parameter sets respectively define different electrode combinations, and may further define different amplitudes for the electrode combinations, such as, e.g., fractionalized electrical current values.

The method further comprises stepping through the stimulation parameter sets of the elected navigation table(s), conveying electrical stimulation energy to the stimulation leads in accordance with the stepped through stimulation parameter sets, and selecting at least one stimulation parameter set (e.g., one of the stepped through stimulation parameter sets) based on the effectiveness of the conveyed electrical stimulation energy. In method, each of the leads carries a plurality of electrodes, and the electrical stimulation energy conveyed to the stimulation leads in accordance with the stepped through stimulation parameter sets results in the shifting of electrical current between the electrodes of the leads. The method further comprises programming the control device with the selected stimulation parameter set(s).

In accordance with a second aspect of the present inventions, a computer readable medium for programming a control device is provided. The control device is configured for controlling electrical stimulation energy provided to a plurality of electrode leads that are physically implanted within a patient in a side-by-side lead configuration. The computer medium contains instructions, which when executed, comprises allowing one of a plurality of different lead stagger configurations to be selected, selecting at least one navigation table that corresponds to the selected lead configuration from a plurality of different navigation tables, stepping through the stimulation parameter sets of selected navigation table(s), and selecting at least one stimulation parameter set for programming the control device. The details of these steps can be the same as those described above with respect to the first aspect of the present inventions.

In accordance with a third aspect of the present inventions, a tissue stimulation system is provided. The system comprises a plurality of electrode leads configured for being placed adjacent tissue of a patient in a side-by-side configuration and an implantable device (e.g., an implantable pulse generator) configured for conveying electrical stimulation energy to the electrode leads to stimulate the tissue. The system further comprises a programming device, such as, e.g., a computer or a hand-held remote control.

The programming device is configured for allowing one of a plurality of different lead stagger configurations to be selected, selecting at least one navigation table that corresponds to the selected lead configuration from a plurality of different navigation tables, stepping through the stimulation parameters sets of the selected navigation table(s), and transmitting the stepped through stimulation parameter sets to the implantable device, wherein the implantable device is configured for conveying the electrical stimulation energy in accordance the stepped through stimulation parameter sets. The programming device is further configured for selecting at least one stimulation parameter set, and programming the implantable device with the selected stimulation parameter set(s). The details of programming device functions can be the same as those described above with respect to the method.

In accordance with a fourth aspect of the present inventions, another method of programming a control device is provided. The control device is configured for controlling electrical stimulation energy provided to a plurality of electrode leads that are physically implanted within a patient (e.g., adjacent a spinal cord of the patient) in a side-by-side lead configuration. The control device may be, e.g., an implantable pulse generator, an external trial stimulator, or an external device configured for controlling the electrical stimulation energy output by the implantable device to the electrode leads.

The method comprises conveying electrical energy from the electrode leads to create a stimulation region within the patient, and automatically shifting the stimulation region along the electrode leads in accordance with an electrical current shifting pattern that is based on a stagger of the side-by-side lead configuration. The electrical current shifting pattern may be defined by any means, such as at least one navigation table or computationally. As examples, the stimulation region may be automatically shifted along the electrode leads in accordance with a first electrical current shifting pattern if the side-by-side lead configuration is a non-staggered lead configuration and a second electrical current shifting pattern if the side-by-side lead configuration is a staggered lead configuration, or the stimulation region may be automatically shifted along the electrode leads in accordance with a first electrical current shifting pattern if the side-by-side lead configuration is a first staggered lead configuration and a second electrical current shifting pattern if the side-by-side lead configuration is a second staggered lead configuration. In one method, the stimulation region is automatically shifted along the electrode leads such that a cathode on one of the leads is never next to an anode on another of the leads.

The method further comprises selecting at least one stimulation parameter set based on the effectiveness of the shifted stimulation region, and programming the control device with the at least one selected stimulation parameter set. In one method, the stimulation parameter sets respectively define different electrode combinations, and may further define different amplitudes for the electrode combinations, such as, e.g., fractionalized electrical current values.

In accordance with a fifth aspect of the present inventions, a tissue stimulation system is provided. The system comprises a plurality of electrode leads configured for being placed adjacent tissue of a patient in a side-by-side configuration and an implantable device (e.g., an implantable pulse generator) configured for conveying electrical stimulation energy to the electrode leads to create a stimulation region within the tissue. The system further comprises a programming device, such as, e.g., a computer or a hand-held remote control.

The programming device is configured for automatically shifting the stimulation region along the electrode leads in accordance with an electrical current shifting pattern that is based on a stagger of the side-by-side lead configuration, selecting at least one stimulation parameter set, and programming the implantable device with the selected stimulation parameter set(s). The details of programming device functions can be the same as those described above with respect to the method.

In accordance with a sixth aspect of the present inventions, a method of selecting one of a plurality of different side-by-side lead stagger configurations corresponding to an actual lead stagger configuration of electrode leads implanted adjacent tissue (e.g., spinal cord tissue) within a patient in a side-by-side configuration is provided. The method comprises displaying a graphical representation of at least one lead stagger configuration, and selecting one of the different lead stagger configurations by interacting with the displayed graphical representation of the lead stagger configuration(s). In one method, a plurality of different lead stagger configurations is simultaneously displayed. In this case, the step of selecting one of the lead stagger configurations may comprise clicking on one of the lead stagger configurations in the graphical representation. In another method, the step of selecting one of the lead stagger configurations comprises incrementally shifting one of the leads relative to another one of the leads (e.g., by clicking on a graphical arrow) in the graphical representation.

The method further comprises performing a function with reference to the selected lead stagger configuration. For example, the function may comprise conveying electrical energy from the actual leads to create a stimulation region within the tissue of the patient as the selected lead stagger configuration is graphically displayed. The stimulation region may be moved relative to the actual leads as the selected lead stagger configuration is graphically displayed. As another example, the graphical representation of the selected lead stagger configuration may include electrodes, in which case, the function may comprise displaying stimulation parameters (e.g., fractionalized electrical current values) adjacent the graphical representations of the electrodes. As still another example, the function may comprise programming a control device configured controlling electrical stimulation energy provided to the actual electrode leads based on the selected lead stagger configuration

In accordance with a seventh aspect of the present inventions, a computer readable medium for programming a control device. The control device is configured for controlling electrical stimulation energy provided to a plurality of electrode leads that are physically implanted within a patient in a side-by-side lead configuration. The computer medium contains instructions, which when executed, comprises displaying a graphical representation of at least one lead stagger configuration, allowing a user to select one of the different lead stagger configurations by interacting with the displayed graphical representation of the at least one lead stagger configuration, and performing a function with reference to the selected lead stagger configuration. The details of these steps can be the same as those described above with respect to the sixth aspect of the present inventions.

In accordance with an eighth aspect of the present inventions, a tissue stimulation system is provided. The system comprises a plurality of electrode leads configured for being placed adjacent tissue of a patient in a side-by-side configuration and an implantable device (e.g., an implantable pulse generator) configured for conveying electrical stimulation energy to the electrode leads to stimulate the tissue. The system further comprises a programming device, such as, e.g., a computer or a hand-held remote control.

The programming device is configured for displaying a graphical representation of at least one lead stagger configuration, allowing a user to select one of the different lead stagger configurations by interacting with the displayed graphical representation of the at least one lead stagger configuration, and performing a function with reference to the selected lead stagger configuration. The details of programming device functions can be the same as those described above with respect to the method.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is perspective view of one embodiment of a SCS system arranged in accordance with the present inventions;

FIG. 2 is a plan view of the SCS system of FIG. 1 in use with a patient;

FIG. 3 is a side view of an implantable pulse generator and a pair of stimulation leads that can be used in the SCS system of FIG. 1;

FIG. 4 is a plan view of a remote control that can be used in the SCS system of FIG. 1;

FIG. 5 is a block diagram of the internal componentry of the remote control of FIG. 4;

FIG. 6 is a block diagram of the components of a computerized programming system that can be used in the SCS system of FIG. 1;

FIG. 7 is a first operating room mapping screen that can be displayed by the computerized programming system of FIG. 6;

FIG. 8 is a second operating room mapping screen that can be displayed by the computerized programming system of FIG. 6, particularly showing a first fractionalized electrode configuration in the E-Troll mode;

FIG. 9 is a third operating room mapping screen that can be displayed by the computerized programming system of FIG. 6, particularly showing a second fractionalized electrode configuration in the E-troll mode;

FIG. 10 is a fourth operating room mapping screen that can be displayed by the computerized programming system of FIG. 6, particularly showing a third fractionalized electrode configuration in the E-troll mode;

FIG. 11 is a first navigator programming screen that can be displayed by the computerized programming system of FIG. 6;

FIG. 12 is a second navigator programming screen that can be displayed by the computerized programming system of FIG. 6, particularly showing a fractionalized electrode configuration;

FIG. 13 is a third navigator programming screen that can be displayed by the computerized programming system of FIG. 6, particularly showing the creation of four marks and corresponding stimulation regions;

FIG. 14 is a coverage areas screen that can be displayed by the computerized programming system of FIG. 6;

FIG. 15 is a lead stagger selection screen that can be displayed by the computerized programming system of FIG. 6;

FIG. 16 is a portion of a first navigation table containing different fractionalized electrode combinations that can be used by the computerized programming system of FIG. 6 to steer current within a pair of electrode leads when implanted in a side-by-side configuration having a first lead stagger;

FIG. 17 is a portion of a second navigation table containing different fractionalized electrode combinations that can be used by the computerized programming system of FIG. 6 to steer current within a pair of electrode leads when implanted in a side-by-side configuration having a second lead stagger;

FIG. 18 is a portion of a third navigation table containing different fractionalized electrode combinations that can be used by the computerized programming system of FIG. 6 to steer current within a pair of electrode leads when implanted in a side-by-side configuration having a third lead stagger;

FIG. 19 is a portion of a fourth navigation table containing different fractionalized electrode combinations that can be used by the computerized programming system of FIG. 6 to steer current within a pair of electrode leads when implanted in a side-by-side configuration having a fourth lead stagger;

FIG. 20 is a portion of a fifth navigation table containing different fractionalized electrode combinations that can be used by the computerized programming system of FIG. 6 to steer current within a pair of electrode leads when implanted in a side-by-side configuration having a fifth lead stagger;

FIG. 21 is a first fractionalized electrode configuration that can be created with the navigation table of FIG. 16;

FIG. 22 is a second fractionalized electrode configuration that can be created with the navigation table of FIG. 16;

FIG. 23 is a third fractionalized electrode configuration that can be created with the navigation table of FIG. 17;

FIG. 24 is a fourth fractionalized electrode configuration that can be created with the navigation table of FIG. 17;

FIG. 25 is a fifth fractionalized electrode configuration that can be created with the navigation table of FIG. 18;

FIG. 26 is a sixth fractionalized electrode configuration that can be created with the navigation table of FIG. 18;

FIG. 27 is a fifth fractionalized electrode configuration that can be created with the navigation table of FIG. 19;

FIG. 28 is a sixth fractionalized electrode configuration that can be created with the navigation table of FIG. 19;

FIG. 29 is a fifth fractionalized electrode configuration that can be created with the navigation table of FIG. 20;

FIG. 30 is a sixth fractionalized electrode configuration that can be created with the navigation table of FIG. 20; and

Appendix A is an exemplary navigation table containing different fractionalized electrode combinations that can be used in a spinal cord stimulation (SCS) system.

### DETAILED DESCRIPTION OF THE EMBODIMENTS

The description that follows relates to a spinal cord stimulation (SCS) system. However, it is to be understood that the while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

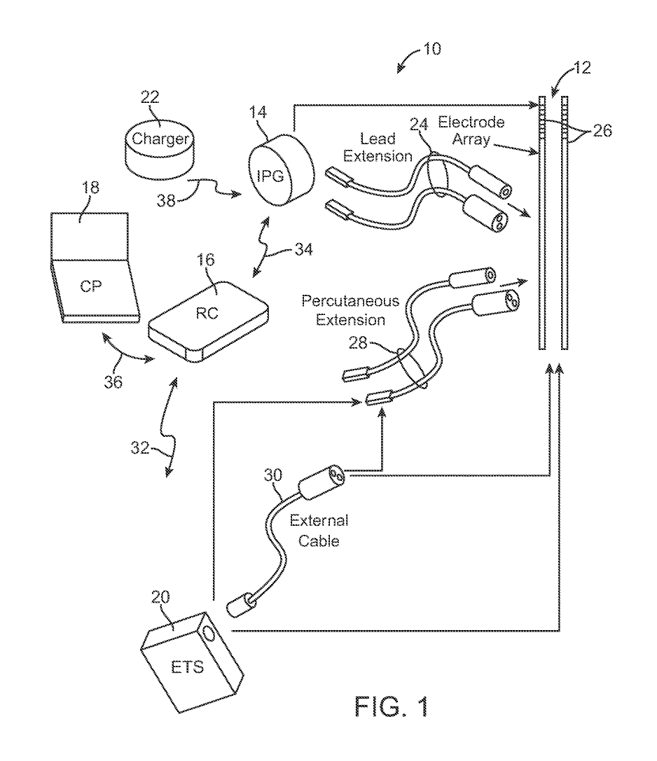

Turning first to FIG. 1, an exemplary SCS system 10 generally includes a plurality (in this case, two) of implantable stimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the stimulation leads 12, which carry a plurality of electrodes 26 arranged in an array. In the illustrated embodiment, the stimulation leads 12 are percutaneous leads, and to this end, the electrodes 26 are arranged in-line along the stimulation leads 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the stimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the stimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and stimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

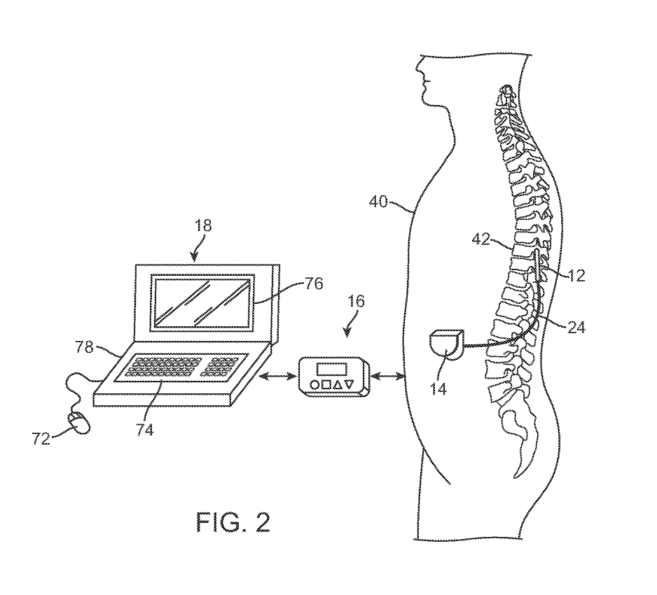

As shown in FIG. 2, the electrode leads 12 are implanted within the spinal column 42 of a patient 40. The preferred placement of the electrode leads 12 is adjacent, i.e., resting upon, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extension 24 facilitates locating the IPG 14 away from the exit point of the electrode leads 12. As there shown, the CP 18 communicates with the IPG 14 via the RC 16.

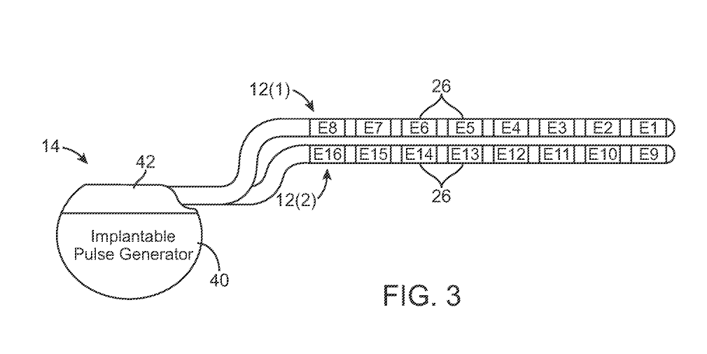

Referring now to FIG. 3, the external features of the stimulation leads 12 and the IPG 14 will be briefly described. One of the stimulation leads 12(1) has eight electrodes 26 (labeled E1-E8), and the other stimulation lead 12(2) has eight electrodes 26 (labeled E9-E16). The actual number and shape of leads and electrodes will, of course, vary according to the intended application. The IPG 14 comprises an outer case 40 for housing the electronic and other components (described in further detail below), and a connector 42 to which the proximal ends of the stimulation leads 12 mates in a manner that electrically couples the electrodes 26 to the electronics within the outer case 40. The outer case 40 is composed of an electrically conductive, biocompatible material, such as titanium, and forms a hermetically sealed compartment wherein the internal electronics are protected from the body tissue and fluids. In some cases, the outer case 40 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the case of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the stimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

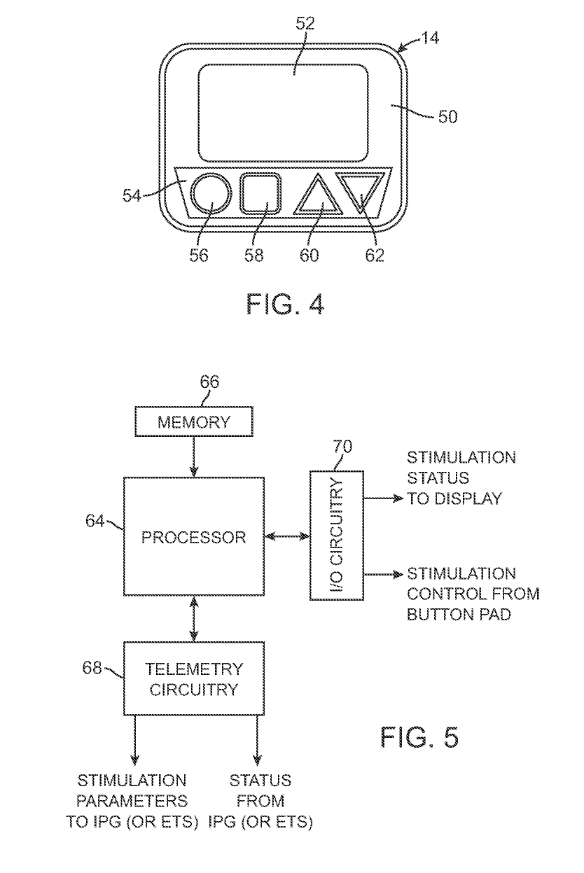

Referring now to FIG. 4, one exemplary embodiment of an RC 16 will now be described. As previously discussed, the RC 16 is capable of communicating with the IPG 14, CP 18, or ETS 20. The RC 16 comprises a casing 50, which houses internal componentry (including a printed circuit board (PCB)), and a lighted display screen 52 and button pad 54 carried by the exterior of the casing 50. In the illustrated embodiment, the display screen 52 is a lighted flat panel display screen, and the button pad 54 comprises a membrane switch with metal domes positioned over a flex circuit, and a keypad connector connected directly to a PCB. In an optional embodiment, the display screen 52 has touchscreen capabilities. The button pad 54 includes a multitude of buttons 56, 58, 60, and 62, which allow the IPG 14 to be turned ON and OFF, provide for the adjustment or setting of stimulation parameters within the IPG 14, and provide for selection between screens.

In the illustrated embodiment, the button 56 serves as an ON/OFF button that can be actuated to turn the IPG 14 ON and OFF. The button 58 serves as a select button that allows the RC 16 to switch between screen displays and/or parameters. The buttons 60 and 62 serve as up/down buttons that can be actuated to increment or decrement any of stimulation parameters of the pulse generated by the IPG 14, including pulse amplitude, pulse width, and pulse rate. For example, the selection button 58 can be actuated to place the RC 16 in a “Pulse Amplitude Adjustment Mode,” during which the pulse amplitude can be adjusted via the up/down buttons 60, 62, a “Pulse Width Adjustment Mode,” during which the pulse width can be adjusted via the up/down buttons 60, 62, and a “Pulse Rate Adjustment Mode,” during which the pulse rate can be adjusted via the up/down buttons 60, 62. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 5, the internal components of an exemplary RC 16 will now be described. The RC 16 generally includes a processor 64 (e.g., a microcontroller), memory 66 that stores an operating program for execution by the processor 64, as well as stimulation parameter sets in a navigation table (described below), input/output circuitry, and in particular, telemetry circuitry 68 for outputting stimulation parameters to the IPG 14 and receiving status information from the IPG 14, and input/output circuitry 70 for receiving stimulation control signals from the button pad 54 and transmitting status information to the display screen 52 (shown in FIG. 4). As well as controlling other functions of the RC 16, which will not be described herein for purposes of brevity, the processor 64 generates new stimulation parameter sets in response to the user operation of the button pad 54. These new stimulation parameter sets would then be transmitted to the IPG 14 (or ETS 20) via the telemetry circuitry 68. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the user (e.g., the physician or clinician) to readily determine the desired stimulation parameters to be programmed into the IPG 14, as well as the RC 16. Thus, modification of the stimulation parameters in the programmable memory of the IPG 14 after implantation is performed by a user using the CP 18, which can directly communicate with the IPG 14 or indirectly communicate with the IPG 14 via the RC 16. That is, the CP 18 can be used by the user to modify operating parameters of the electrode array 26 near the spinal cord.

As shown in FIG. 2, the overall appearance of the CP 18 is that of a laptop personal computer (PC), and in fact, may be implanted using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

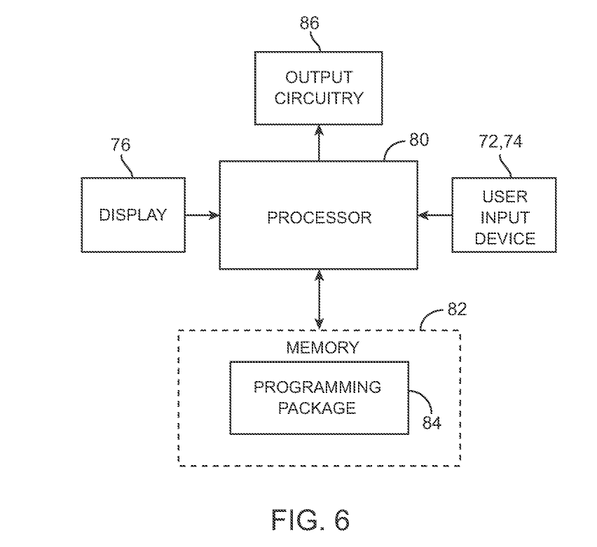

To allow the user to perform these functions, the CP 18 includes a mouse 72, a keyboard 74, and a programming display screen 76 housed in a case 78. It is to be understood that in addition to, or in lieu of, the mouse 72, other directional programming devices may be used, such as a joystick, or directional keys included as part of the keys associated with the keyboard 74. As shown in FIG. 6, the CP 18 generally includes a processor 80 (e.g., a central processor unit (CPU)) and memory 82 that stores a stimulation programming package 84, which can be executed by the processor 80 to allow the user to program the IPG 14, and RC 16. The CP 18 further includes output circuitry 86 (e.g., via the telemetry circuitry of the RC 16) for downloading stimulation parameters to the IPG 14 and RC 16 and for uploading stimulation parameters already stored in the memory 66 of the RC 16, via the telemetry circuitry 68 of the RC 16.

Execution of the programming package 84 by the processor 80 provides a multitude of display screens (not shown) that can be navigated through via use of the mouse 72. These display screens allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, and define the configuration and orientation of the leads, initiate and control the electrical stimulation energy output by the leads 12, and select and program the IPG 14 with stimulation parameters in both a surgical setting and a clinical setting.

As will be described in further detail below, the processor 80 provides display screens that allow a user to convey the electrical energy from the leads 12 to create a stimulation region within the patient, automatically shift the stimulation region along the leads 12 in accordance with an electrical shifting pattern, selecting at least one stimulation parameter set based on the effectiveness of the shifted stimulation region, and programming the IPG 14 with the stimulation parameter set(s). Further details discussing the above-described CP functions are disclosed in U.S. Provisional Patent Application Ser. No. 61/080,187, entitled “System and Method for Converting Tissue Stimulation Programs in a Format Usable by an Electrical Current Steering Navigator,” which is expressly incorporated herein by reference.

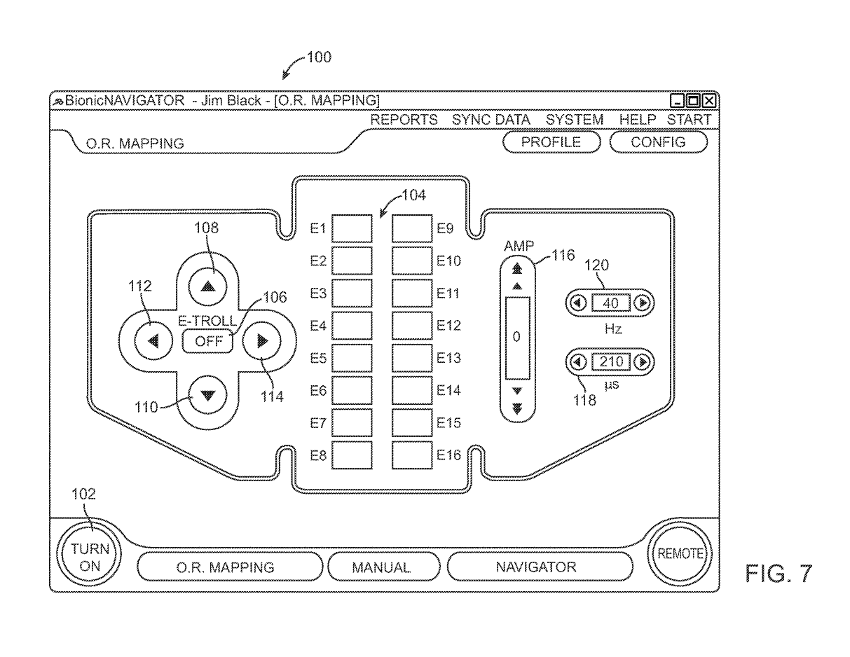

In the context of an operating room procedure, execution of the programming package 84 may open an OR mapping screen 100, as shown in FIG. 7, which allows a clinician to assess lead position and evaluate paresthesia coverage during surgery via an Electronic Trolling (E-Troll) function. E-Troll is a quick way to sweep the electrode array by gradually moving a cathode in bipolar stimulation. To this end, the OR mapping screen 100 includes an E-Troll button 106 that can be clicked to enable the E-trolling function, and up, down, left, and right arrows 108-114 to respectively move the cathode or cathodes up, down, left and right in the electrode array, thereby steering the electrical current, and thus, the resulting stimulation region, up, down, left, and right in the electrode array, in accordance with an electrical current steering pattern, which in the illustrated embodiment, is defined by a navigation table. Actuation of the power-on button 102 in the OR mapping screen 100 directs the IPG 14 to alternatively deliver or cease delivering stimulation energy to the electrode array 26 (corresponding to the graphical electrode representation 104 shown in FIG. 7) in accordance with the stimulation parameters generated during the E-troll function and transmitted from the CP 18 to the IPG 14 via the RC 16.

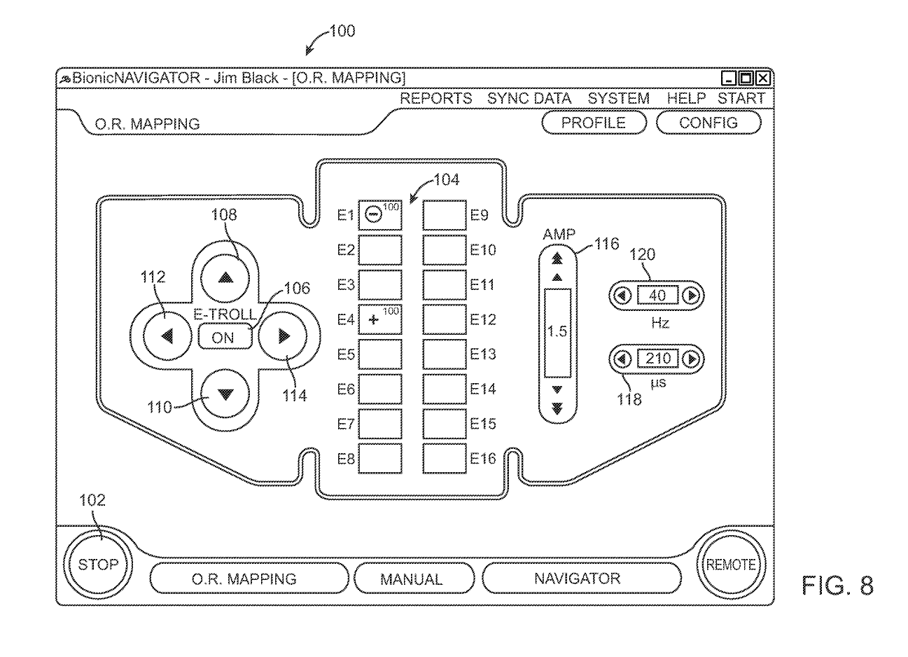

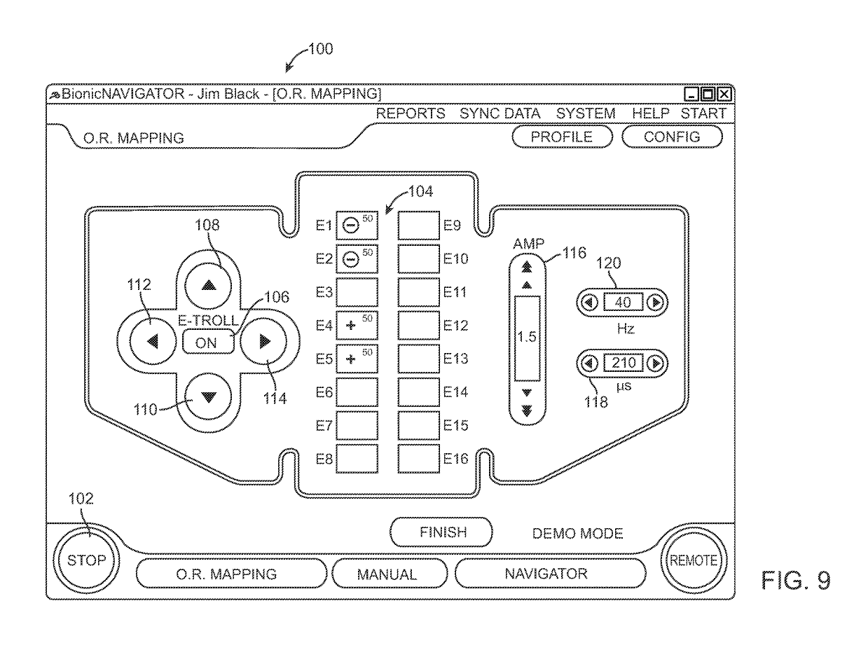

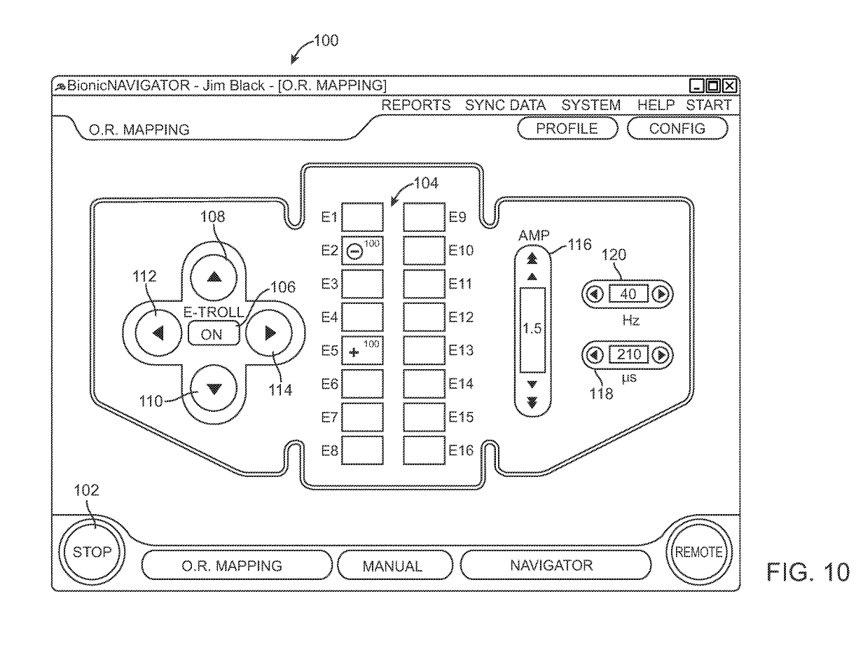

For example, as shown in FIG. 8, the E-Troll process may begin by designating electrode E1 as the sole cathode and electrode E4 as the sole anode. As there shown, electrode E1 has a fractionalized cathodic current value of 100%, and electrode E4 has a fractionalized anodic current value of 100%. If the down button 110 is clicked, the cathodic current is gradually shifted from electrode E1 to electrode E2, and the anodic current is gradually shifted from electrode E4 to electrode E5, which gradual shifting occurs in 10% increments. For example, as shown in FIG. 9, the electrical current is shifted, such that electrode E1 has a fractionalized cathodic current value of 50%, electrode E2 has a fractionalized cathodic current value of 50%, electrode E4 has a fractionalized anodic current value of 50%, and electrode E5 has a fractionalized anodic current value of 50%. As shown in FIG. 10, the electrical current is further shifted, such that electrode E2 has a fractionalized cathodic current value of 100%, and electrode E5 has a fractionalized anodic current value of 100%. Further clicking of the down button 110 shifts the cathodic current and anodic current further down the electrode array in a similar manner. Likewise, clicking the up button 108, left button 112, or right button 114 causes the cathodic currents and anodic currents to respectively shift up, left, and right within the electrode array in a similar manner.

In the illustrated embodiment, a navigation table, such as the one shown in Appendix A, is used to generate fractionalized electrode configurations for each lead 12. Because the navigation table only contains fractionalized electrode configurations for a single lead (i.e., 8 electrodes) to independently generate fractionalized electrode configurations for each lead 12 (one for electrodes E1-E8 and one for electrodes E9-E16), which for purposes of displaying to the clinician in OR mapping screen 100(5), can then be combined into a single fractionalized electrode configuration and normalized, such that the fractionalized cathodic current for both leads 12 (i.e., the entire electrode array 26) totals 100% and the fractionalized anodic current for both leads 12 (i.e., the entire electrode array 26) totals 100%. As will be described in further detail below, during the E-troll function, different navigation tables can be utilized based on the stagger of the leads 12.

The cathodic and anodic currents can be shifted up and down along each lead 12 by stepping up and down through the fractionalized electrode configurations within the navigation table. The cathodic and anodic currents can be shifted left and right by scaling the currents on the first and second leads relative to each other. That is, to steer current from the second lead to the first lead, the fractionalized electrode configuration for the second lead is scaled down, and the fractionalized electrode configuration for the first lead is scaled up, and to steer current from the first lead to the second lead, the fractionalized electrode configuration for the first lead is scaled down, and the fractionalized electrode configuration for the second lead is scaled up.

The OR mapping screen 100, as shown in FIG. 10, also allows the clinician to modify the stimulation energy (i.e., the electrical pulse parameters) output by the IPG 14 to the electrodes during the E-troll function by adjusting each of a pulse amplitude, pulse width, or pulse rate. To this end, OR mapping screen 100 includes a pulse amplitude adjustment control 116, the top arrow of which can be clicked to incrementally increase the pulse amplitude of the stimulation energy, and the bottom arrow of which can be clicked to incrementally decrease the pulse amplitude of the stimulation energy. The OR mapping screen 100 further includes a pulse width adjustment control 118, the right arrow of which can be clicked to incrementally increase the pulse width of the stimulation energy, and the left arrow of which can be clicked to incrementally decrease the pulse width of the stimulation energy. The OR mapping screen 100 further includes a pulse rate adjustment control 120, the right arrow of which can be clicked to incrementally increase the pulse rate of the stimulation energy, and the left arrow of which can be clicked to incrementally decrease the pulse rate of the stimulation energy. Notably, the adjustment of the pulse amplitude, pulse width, and pulse rate will be performed globally for all of the electrodes activated as either an anode (+) or a cathode (−).

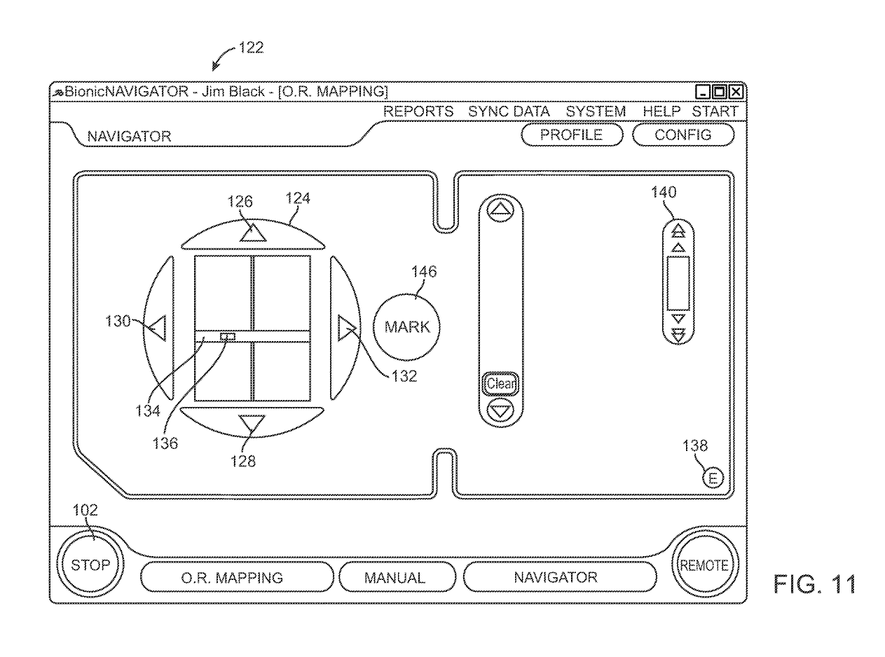

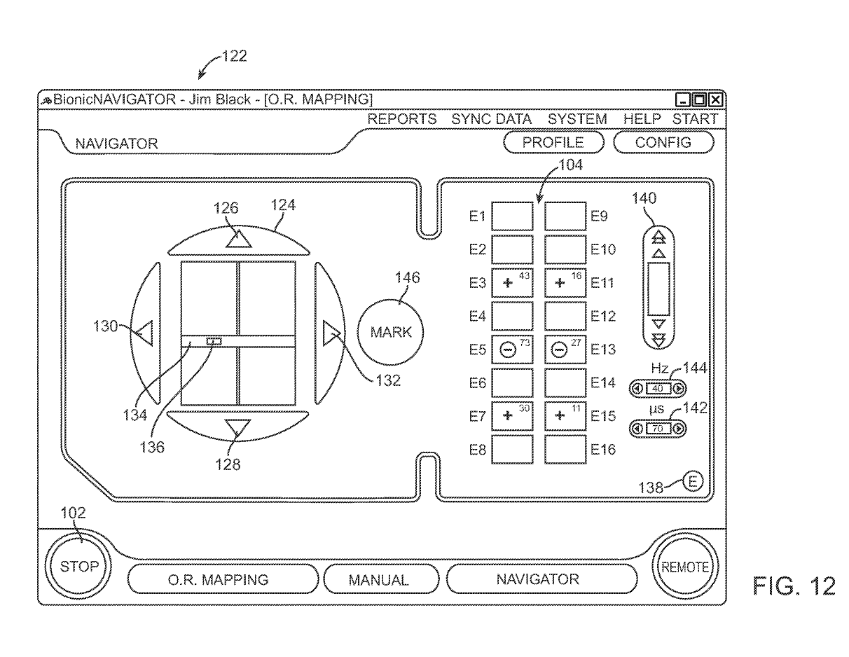

In the context of a follow-up procedure, execution of the programming package 84 may open up a navigator screen 122 that allows a clinician to shift current between multiple electrode combinations to fine tune and optimize stimulation coverage for patient comfort, as shown in FIG. 11. To this end, the navigator screen 122 includes a navigator scope 124 that represents the stimulation region along the spinal cord relative to the electrode array that can be targeted using directional controls 126-132 (up, down, left, and right arrows). The navigator scope 124 has a horizontal bar 134 with a location designator (represented by a rectangular opening) 136 that indicates the current location of the stimulation region relative to the electrode array. Clicking on the up and down control arrows 126, 128 displaces the horizontal bar 134, and thus the location designator 136, up and down within the navigator scope 124, and clicking on the left and right control arrows 130, 132 displaces the location designator 136 left and right along the horizontal bar 134. Thus, the stimulation region can be displaced upward by clicking on the up control arrow 126, displaced downward by clicking on the down control arrow 128, displaced to the left by clicking on the left control arrow 130, and displaced to the right by clicking on the right control arrow 132. Notably, actuation of the power-on button 102 in the navigator screen 122 directs the IPG 14 to alternatively deliver or cease delivering stimulation energy to the electrode array 26 (corresponding to the graphical electrode representation 104 shown in FIG. 12) in accordance with the stimulation parameters generated during the navigation function and transmitted from the CP 18 to the IPG 14 via the RC 16.

Significantly, the navigator scope 124 displaces the stimulation region by steering the electrical current (i.e., shifting electrical current between the electrodes E1-E16) in a manner similar to that used by the E-Troll function described above to shift current between the electrodes E1-E16. Thus, clicking the up control arrow 126 displaces the cathode upward in the electrode array, thereby displacing the stimulation region upward relative the spinal cord; clicking the down control arrow 128 displaces the cathode downward in the electrode array, thereby displacing the stimulation region downward relative to the spinal cord; clicking the left control arrow 130 displaces the cathode to the left in the electrode array, thereby displacing the stimulation region to the left relative to the spinal cord; and clicking the right control arrow 132 displaces the cathode to the right in the electrode array, thereby displacing the stimulation region to the right relative to the spinal cord.

In the illustrated embodiment, a navigation table, such as the one shown in Appendix A, is used to generate fractionalized electrode configurations for each lead 12. Again, because the navigation table only contains fractionalized electrode configurations for a single lead (i.e., 8 electrodes), two identical navigation tables will be used to independently generate fractionalized electrode configurations for each lead 12 (one for electrodes E1-E8 and one for electrodes E9-E16), which for purposes of displaying to the clinician in the navigation 122, can then be combined into a single fractionalized electrode configuration and normalized, such that the fractionalized cathodic current for both leads 12 (i.e., the entire electrode array 26) totals 100% and the fractionalized anodic current for both leads 12 (i.e., the entire electrode array 26) totals 100%. As will be described in further detail below, during the navigation function, different navigation tables can be utilized based on the stagger of the leads 12. The cathodic and anodic currents can be shifted up and down along each lead 12 and shifted left and right between the leads 12 in the same manner described above with respect to the E-Troll function.

The navigator screen 122 also includes an electrode combination button 138 that can be clicked to allow clinician to view the fractionalized electrode configuration 104 that corresponds to the stimulation region identified by the location designator 136, as shown in FIG. 12. As there shown, electrodes E3, E7, E11, and E15 respectively have fractionalized cathodic current values of 43%, 30%, 16%, and 11%, and electrodes E5 and E13 respectively have anodic current values of 73% and 27% to locate the stimulation region at the location currently pointed to by the location designator 136. The navigator screen 122 also allows the clinician to modify the stimulation energy (i.e., the electrical pulse parameters) output by the IPG 14 by adjusting each of a pulse amplitude or a pulse rate.

To this end, the navigator screen 122 includes a pulse amplitude adjustment control 140, the top arrow of which can be clicked to incrementally increase the pulse amplitude of the stimulation energy, and the bottom arrow of which can be clicked to incrementally decrease the pulse amplitude of the stimulation energy. The navigator screen 122 further includes a pulse width adjustment control 142 (provided only in the navigator screen 122 illustrated in FIG. 12), the right arrow of which can be clicked to incrementally increase the pulse width of the stimulation energy, and the left arrow of which can be clicked to incrementally decrease the pulse width of the stimulation energy. Notably, the adjustment of the pulse amplitude, pulse width, and pulse rate will be performed globally for all of the electrodes activated as either an anode (+) or a cathode (−). While the navigator screen 122 does not include a pulse rate adjustment control, it does include a pulse rate display 144 (provided only in the navigator screen 122 illustrated in FIG. 12) that provides the default pulse rate for the system to the clinician.

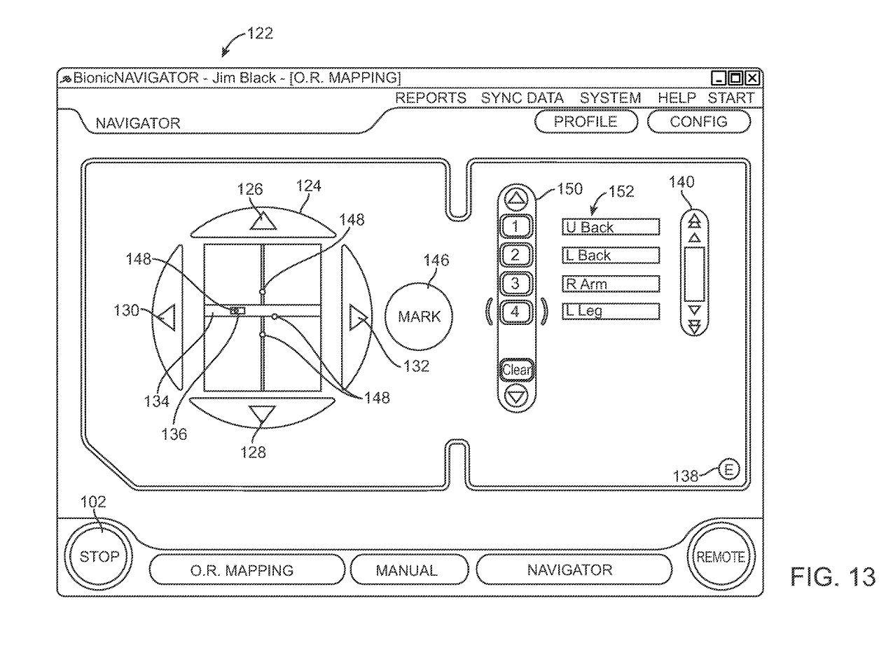

The navigator screen 122 has a mark button 146 that can be clicked to mark points 148 (shown in FIG. 13) where coverage is preferred for the target area; that is, the area that the location designator 136 currently points to when the mark button 146 is clicked will be marked. Each mark 148 is a set of stimulation parameters (including fractionalized electrode configuration, pulse amplitude, pulse width, and pulse rate) that corresponds to the location or area of the stimulation region. As shown in FIG. 13, the navigator screen 122 includes a mark list 150 that includes numbered designators corresponding to all of the marks 148 generated by the navigator scope 124 and an area designator 152 that can be filled in by the clinician to associate an area of paresthesia for each mark 148. As shown in FIG. 13, four marks 148 have been generated, with the first mark being identified as causing paresthesia in the upper back of the patient, the second mark being identified as causing paresthesia in the lower back of the patient, the third mark being identified as causing paresthesia in the right arm of the patient, and the fourth mark being identified as causing paresthesia in the left leg of the patient. Notably, any one of the numbered designated within the mark list 150 can be clicked to center the area designator 136 on the corresponding mark 148 in the navigation scope 124.

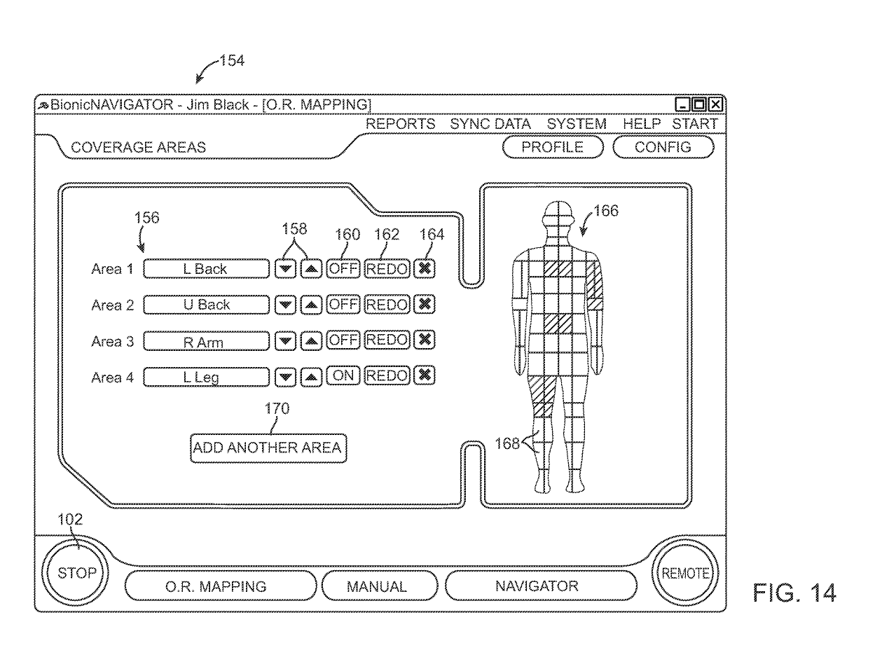

After the marks 148 are generated, execution of the programming package 84 may open up a coverage areas screen 154 that allows the clinician to generate a stimulation program from the marks 148, as shown in FIG. 14. The coverage areas screen 154 includes a list of the coverage areas 156 with corresponding control buttons. In particular, each coverage area 156 has associated with it amplitude up/down arrows 158 that can be clicked to modify the mark corresponding to that coverage area 156 by increasing or decreasing the amplitude of the stimulation energy conveyed by the electrode array 26. Each coverage area 156 also includes an on/off button 160 that can be clicked to alternately provide or cease the delivery of the stimulation energy from the IPG 14 to the electrode array 26. Any combination of the coverage areas 156 can be turned on, so that multiple coverage areas of the patient can be simultaneously stimulated. Each coverage area 156 also includes a redo button 162 that regenerates and stores the mark 148 with any new amplitude values that are adjusted by manipulation of the amplitude up/down arrows 158, and a deletion button 164 that deletes the mark 148 and associated area designation from the coverage areas screen 154.

The coverage areas screen 154 further includes a paresthesia map of the human body 166 divided into several regions 168. Clicking on one or more of these regions 168 allows the clinician to record the regions of paresthesia experienced by the patient for the areas that have been turned on. The paresthesia map 166 also includes regions 168 previously highlighted as indicating pain. Thus, the upper back, lower back, right arm, and left thigh of the patient are highlighted, indicating that these are the regions of pain experienced by the patient. Clicking on any of the regions 168 in the paresthesia map 166 further highlights the regions experienced by the patient as having paresthesia. Any region of paresthesia that corresponds to the same region previously indicated as having pain will be highlighted with a different color (shown hatched). As shown in FIG. 14, the left leg of the patient is highlighted to indicate the region where the patient is experiencing paresthesia when the fourth coverage area 156 is turned on.

The coverage areas screen 154 further includes an add another area button 170 that can be clicked to allow the clinician to add additional marks 148 in the navigator screen 122 of FIG. 13. The groups of stimulation parameter sets can be combined into a single stimulation program that can be transmitted to and stored within the RC 16 and IPG 14 from the CP 18. Further details discussing the generation of stimulation programs from groups of stimulation parameter sets are discussed in U.S. Provisional Patent Application Ser. No. 61/080,187, entitled “System and Method for Converting Tissue Stimulation Programs in a Format Usable by an Electrical Current Steering Navigator,” which has previously been incorporated herein by reference.

Significantly, in the case where the leads 12 are physically implanted within the patient in a side-by-side configuration, the CP 18 allows the electrical current shifting pattern associated with the shifting of the stimulation region to be based on the stagger of the leads 12. For example, the stimulation region may be automatically shifted along the leads 12 in accordance with a first electrical current shifting pattern if the side-by-side lead configuration is a non-staggered lead configuration and a second electrical current shifting pattern if the side-by-side lead configuration is a staggered lead configuration. Or the stimulation region may be automatically shifted along the leads 12 in accordance with a first electrical current shifting pattern if the side-by-side lead configuration is a first staggered lead configuration and a second electrical current shifting pattern if the side-by-side lead configuration is a second staggered lead configuration. Preferably, the stimulation region is automatically shifted along the leads 12 such that a cathode on one of the leads 12 is never next to an anode on another of the leads 12.

In performing this function, the electrical current shifting pattern used by the CP 18 to shift the stimulation region along the leads 12 is defined by one or more navigation tables that are selected in response to an entry of a selected lead stagger configuration corresponding to the actual configuration in which the leads 12 are physically implanted within the patient.

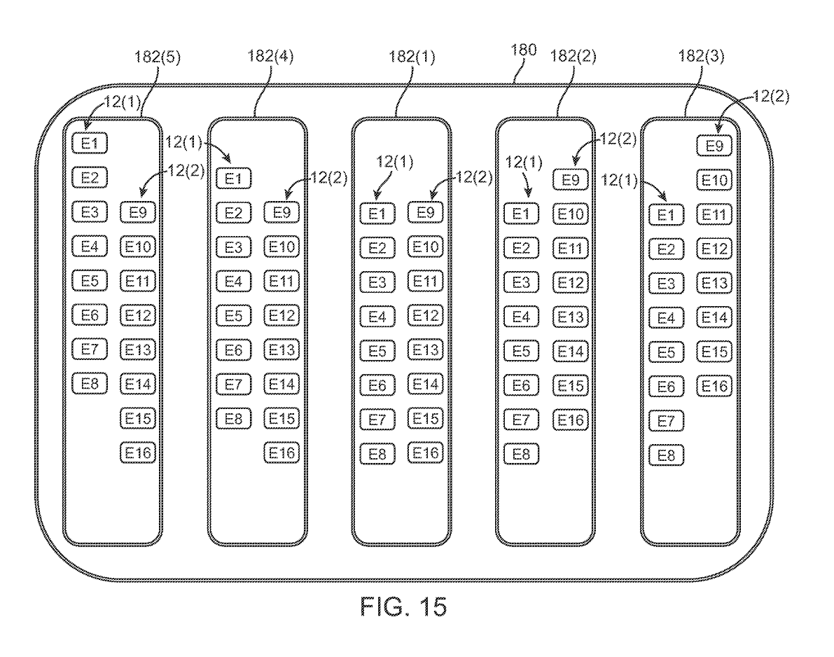

In particular, the execution of the programming package 84 allows the user to select one of a plurality of different lead stagger configurations. For example, referring to FIG. 15, a lead stagger selection screen 180 illustrating graphical representations of a plurality of lead stagger configurations 182(1)-182(5) can be used by the user to select a lead stagger configuration that best matches the stagger of the actual side-by-side configuration of the leads 12. As there shown, the lead stagger configuration 182(1) corresponds to a configuration in which the leads 12(1) and 12(2) have no stagger; the lead stagger configuration 182(2) corresponds to a configuration in which the right lead 12(2) is staggered upward from the left lead 12(1) by one electrode; the lead stagger configuration 182(3) corresponds to a configuration in which the right lead 12(2) is staggered upward from the left lead 12(1) by two electrodes; the lead stagger configuration 182(4) corresponds to a configuration in which the right lead 12(2) is staggered downward from the left lead 12(1) by one electrode; and the lead stagger configuration 182(5) corresponds to a configuration in which the right lead 12(2) is staggered downward from the left lead 12(1) by two electrodes. The user may select any of these lead stagger configurations 182(1)-182(5) by using the mouse 72 to click on the corresponding graphical representation.

Alternatively, the two side-by-side electrodes may be displayed initially as in ‘perfect parallel’, where electrode E1 is laterally adjacent to electrode E9, electrode E2 is laterally adjacent to electrode E10, etc. On the lead stagger selection screen 180, the user is provided with an adjustment control (not shown) that can shift the graphical representation of a selected lead in a manner, such that the graphical representation of the final lead stagger configuration matches the actual stagger configuration of the leads implanted within the body. For example, from the perfect parallel position, the user may select the graphical representation of the right-sided lead on the screen. From there, if the user clicks an “up” arrow on the provided screen control, the graphical representation of the right-sided lead would move upward on the screen by a small amount (e.g., 1 mm) relative to the graphical representation of the static left-sided lead. Repeated clicks would move the graphical representation of the right-sided lead further upwards in 1mm increments, such that the relative stagger of the two leads would increase until the user was satisfied that the displayed graphical representation of the lead stagger configuration matched the stagger configuration of the actual leads implanted in the body.

The user control may also have ‘down,’ ‘left,’ and ‘right’ lead shifting capability. For example, the user control may be provided a “down” arrow that can be repeatedly clicked to incrementally move the graphical representation of a selected lead down relative to the other lead, a “left” arrow that can be repeatedly clicked to incrementally move the graphical representation of a selected lead to the left relative to the other lead, and a “right” arrow that can be repeatedly clicked to incrementally move the graphical representation of a selected lead to the right relative to the other lead.

It should be appreciated that while selection of the lead stagger configurations are described herein as being useful for selecting navigation tables, thereby facilitating current steering, graphical selection of the lead stagger configurations may also lend itself to other applications, such as displaying electrode impedances on the selected lead stagger configuration.

Upon selecting the lead stagger configuration, the CP 18 will select the navigation table corresponding to the selected lead stagger configuration 182(1)-182(5). As discussed above, in the illustrated embodiment, two navigation tables are respectively used for the leads 12, which can then be combined into a single navigation table with normalized fractionalized electrode configurations.

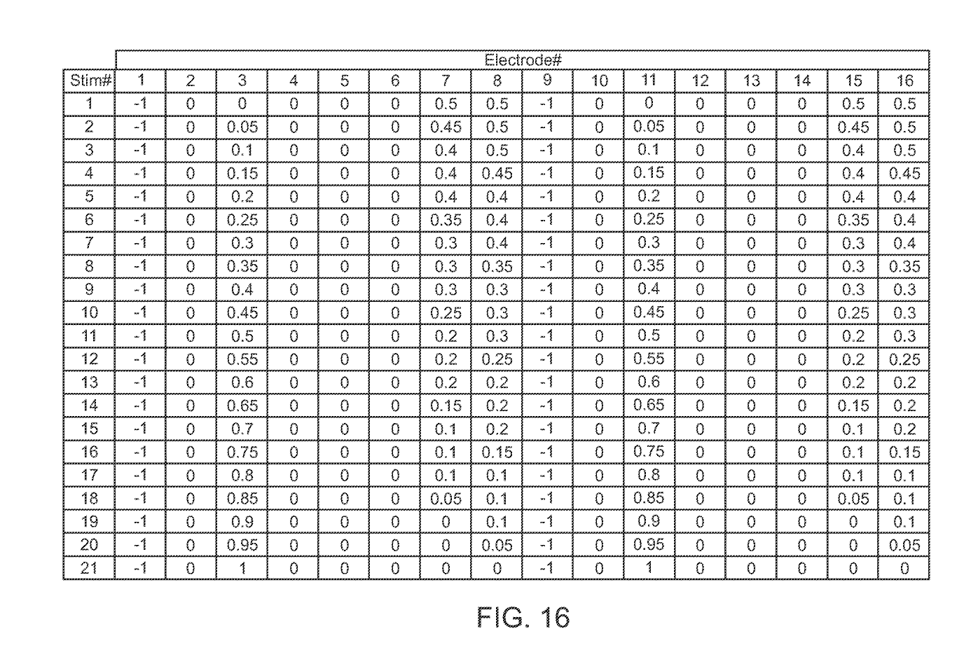

Referring to FIGS. 16-20, portions of five exemplary un-normalized navigation tables that can be selected by the CP 18 in response to the different lead stagger configurations 182(1)-182(5) selected by the user in FIG. 15 will now be described. In these cases, the portions of the navigation tables are defined by stimulation parameter sets 1-21, which define electrode patterns that transition from a first electrode combination that includes a pair cathodes respectively at the top of the leads 12 and two pairs of anodes respectively at the bottom of the leads 12, to a second electrode combination that includes the same pair of cathodes respectively at the top of the lead 12 and a pair of anodes in the middle of the leads 12. Notably, the fractionalized electrode configurations contained in the navigation tables of FIGS. 16-20 are unnormalized, so that, for purposes of illustration, they can be easily compared to the unnormalized fractionalized electrode configurations described below.

In transitioning from the first electrode combination to the second electrode combination, the navigation tables are constructed in a manner that prevents a cathode of one lead 12 to be adjacent to an anode of another lead 12, and preferably, maintains each of the pairs of cathode and anodes in a side-by-side relationship regardless of the stagger between the leads 12.

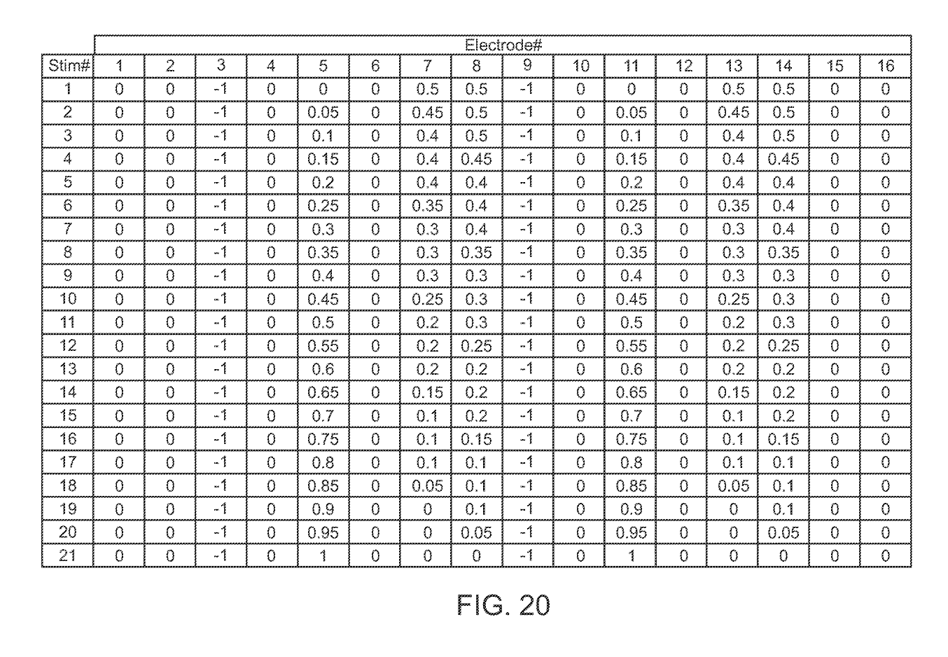

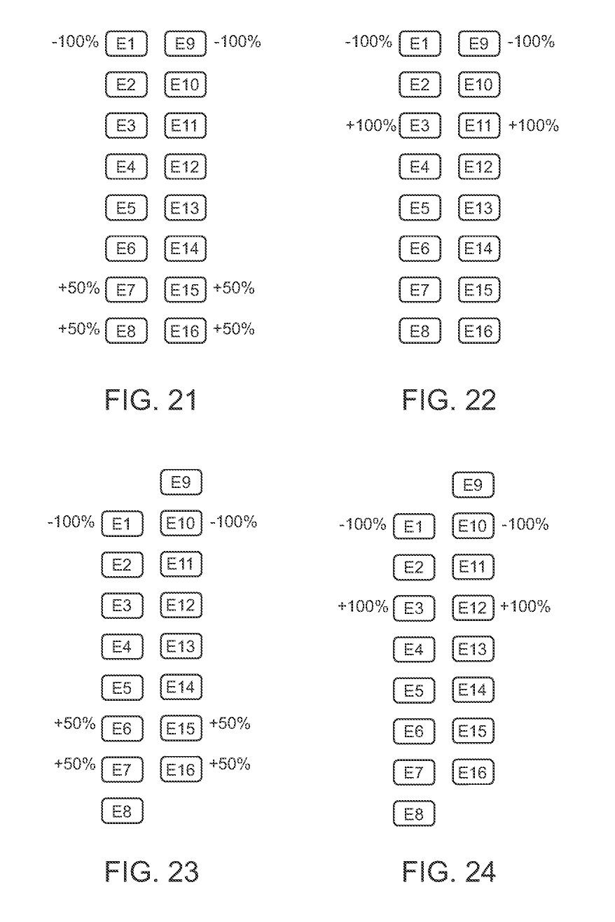

For example, in the case where the leads are in a non-staggered configuration, a nominal navigation table illustrated in FIG. 16 can be used to maintain each of the anode and cathode pairs in a side-by-side relationship as the first fractionalized electrode combination (FIG. 21) transitions to the second fractionalized electrode combination (FIG. 22).

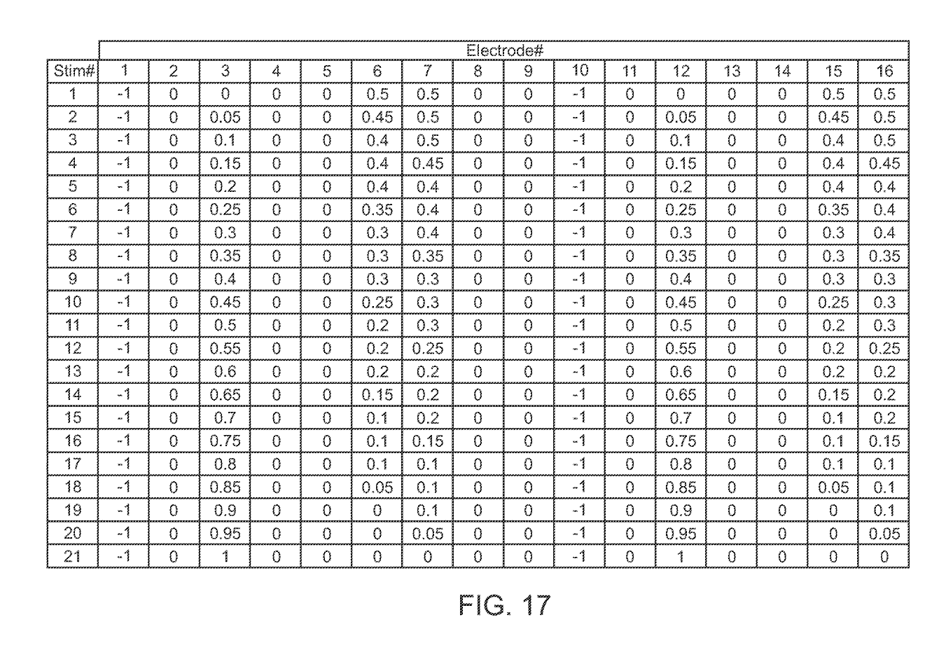

In the case where the right lead 12(2) is staggered upward from the left lead 12(1) by one electrode, the navigation table illustrated in FIG. 17 can be used to maintain each of the anode and cathode pairs in a side-by-side relationship as the first electrode combination (FIG. 23) transitions to the second electrode combination (FIG. 24). Notably, to compensate for this stagger, the fractionalized cathodic values and anodic values respectively associated with electrodes E9 and E11 of the second lead 12(2) in the nominal navigation table of FIG. 16 are respectively shifted downward to electrodes E10 and E12 of the second lead 12(2) in the navigation table of FIG. 17, and the fractionalized anodic values respectively associated with electrodes E7 and E8 of the first lead 12(1) in the nominal navigation table of FIG. 16 are respectively shifted upward to electrodes E6 and E7 of the first lead 12(1) in the navigation table of FIG. 17.

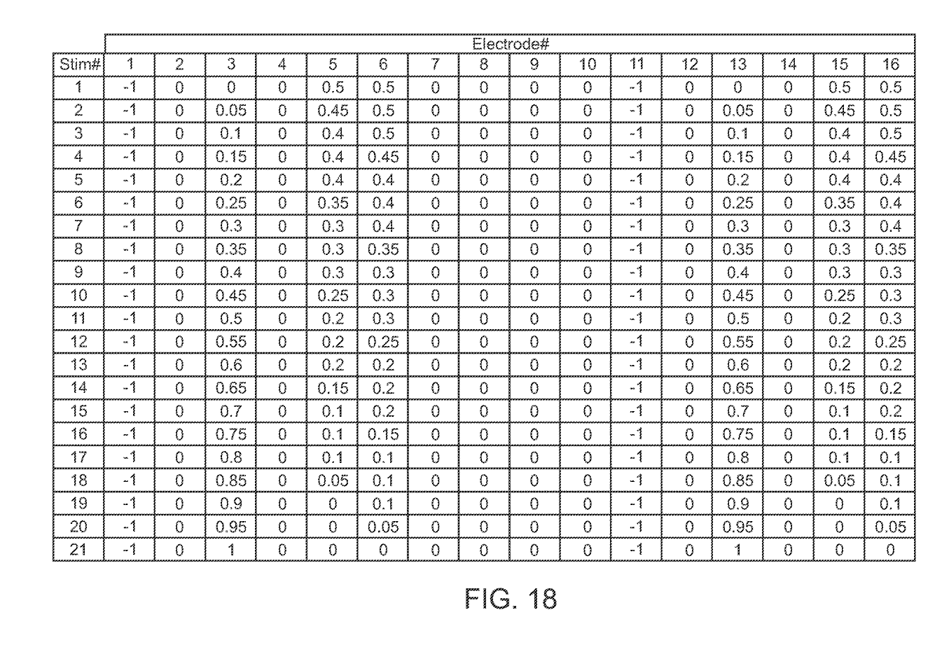

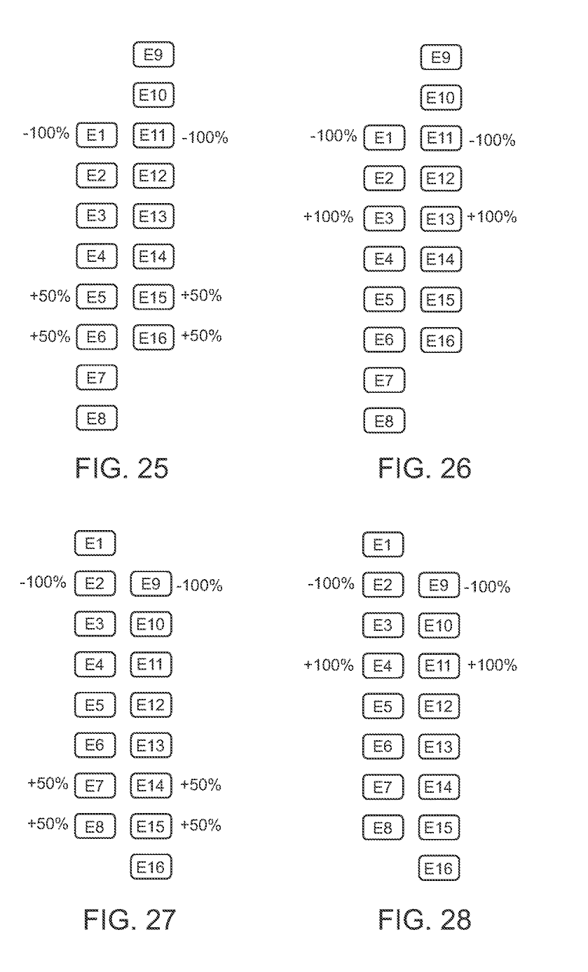

In the case where the right lead 12(2) is staggered upward from the left lead 12(1) by two electrodes, the navigation table illustrated in FIG. 18 can be used to maintain each of the anode and cathode pairs in a side-by-side relationship as the first electrode combination (FIG. 25) transitions to the second electrode combination (FIG. 26). Notably, to compensate for this stagger, the fractionalized cathodic values and anodic values respectively associated with electrodes E9 and E11 of the second lead 12(2) in the nominal navigation table of FIG. 16 are respectively shifted downward to electrodes E11 and E13 of the second lead 12(2) in the navigation table of FIG. 18, and the fractionalized anodic values respectively associated with electrodes E7 and E8 of the first lead 12(1) in the nominal navigation table of FIG. 16 are respectively shifted upward to electrodes E5 and E6 of the first lead 12(1) in the navigation table of FIG. 18.

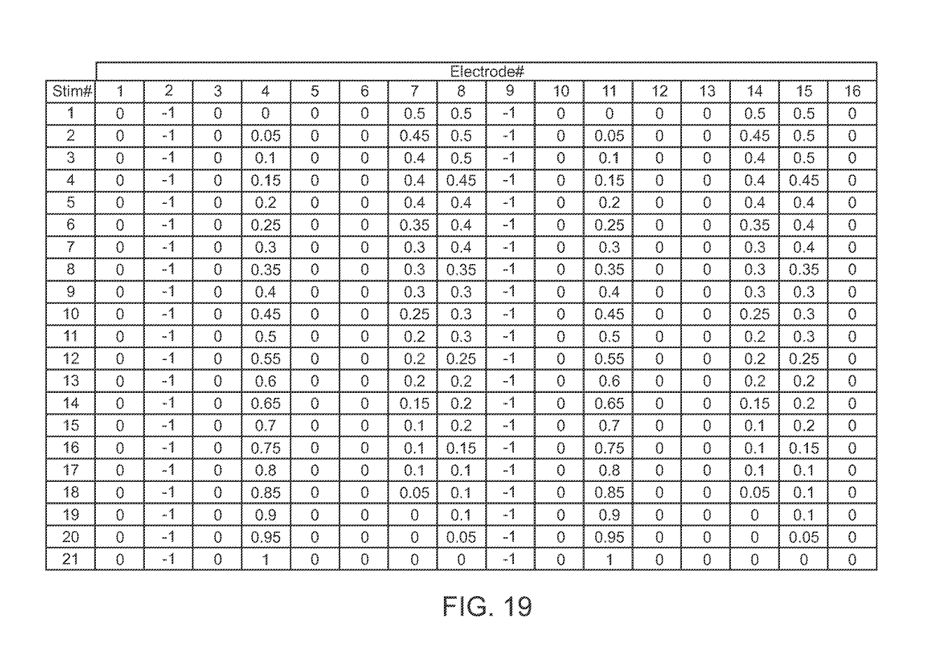

In the case where the right lead 12(2) is staggered downward from the left lead 12(1) by one electrode, the navigation table illustrated in FIG. 19 can be used to maintain each of the anode and cathode pairs in a side-by-side relationship as the first electrode combination (FIG. 27) transitions to the second electrode combination (FIG. 28). Notably, to compensate for this stagger, the fractionalized cathodic values and anodic values respectively associated with electrodes E1 and E3 of the first lead 12(1) in the nominal navigation table of FIG. 16 are respectively shifted downward to electrodes E2 and E4 of the first lead 12(1) in the navigation table of FIG. 19, and the fractionalized anodic values respectively associated with electrodes E15 and E16 of the second lead 12(2) in the nominal navigation table of FIG. 16 are respectively shifted upward to electrodes E14 and E15 of the second lead 12(2) in the navigation table of FIG. 19.

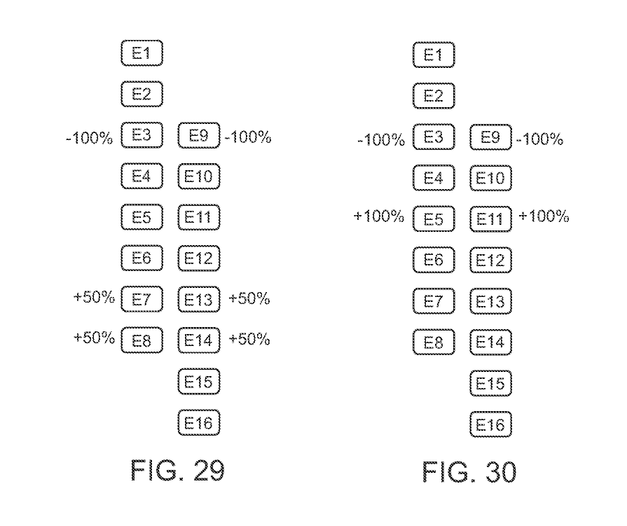

In the case where the right lead 12(2) is staggered downward from the left lead 12(1) by two electrodes, the navigation table illustrated in FIG. 20 can be used to maintain each of the anode and cathode pairs in a side-by-side relationship as the first electrode combination (FIG. 29) transitions to the second electrode combination (FIG. 30). Notably, to compensate for this stagger, the fractionalized cathodic values and anodic values respectively associated with electrodes E1 and E3 of the first lead 12(1) in the nominal navigation table of FIG. 16 are respectively shifted downward to electrodes E3 and E5 of the first lead 12(1) in the navigation table of FIG. 20, and the fractionalized anodic values respectively associated with electrodes E15 and E16 of the second lead 12(2) in the nominal navigation table of FIG. 16 are respectively shifted upward to electrodes E13 and E14 of the second lead 12(2) in the navigation table of FIG. 20.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A tissue stimulation system, comprising:
a plurality of electrode leads configured to be placed adjacent tissue of a patient in a side-by-side configuration;
an implantable device configured to convey electrical stimulation energy to the electrode leads to stimulate the tissue;
a programming device configured to:
display a graphical representation of at least one lead stagger configuration;
allow a user to select one of the different lead stagger configurations by interacting with the displayed graphical representation of the at least one lead stagger configuration; and
perform a function with reference to the selected lead stagger configuration.

2. The system of claim 1, wherein the programming device is configured to simultaneously display a plurality of different lead stagger configurations, and is configured to allow the user to select one of the lead stagger configurations.

3. The system of claim 1, wherein the programming device is configured to allow the user to select one of the lead stagger configurations by incrementally shifting one of the leads relative to another one of the leads in the graphical representation of the at least one lead stagger configuration.

4. The system of claim 1, wherein the function comprises conveying electrical energy from the actual leads to create a stimulation region within the tissue of the patient as the selected lead stagger configuration is graphically displayed.

5. The system of claim 4, wherein the function comprises moving the stimulation region relative to the actual leads as the selected lead stagger configuration is graphically displayed.

6. The system of claim 1, wherein the graphical representation of the selected lead stagger configuration includes electrodes.

7. The system of claim 6, wherein the function comprises displaying stimulation parameters adjacent the graphical representations of the electrodes.

8. The system of claim 7, wherein the displayed stimulation parameters include fractionalized electrical current values.

9. The system of claim 1, wherein the function comprises programming a control device configured to control electrical stimulation energy provided to the actual electrode leads based on the selected lead stagger configuration.

10. A method of selecting one of a plurality of different side-by-side lead stagger configurations corresponding to an actual lead stagger configuration of electrode leads implanted adjacent tissue within a patient in a side-by-side configuration, comprising:
displaying a graphical representation of at least one lead stagger configuration;
selecting one of the different lead stagger configurations by interacting with the displayed graphical representation of the at least one lead stagger configuration; and
performing a function with reference to the selected lead stagger configuration.

11. The method of claim 10, wherein displaying the graphical representation of the at least one lead stagger configuration comprises simultaneously displaying a plurality of different lead stagger configurations, and selecting includes selecting one of the plurality of different lead stagger configurations.

12. The method of claim 10, wherein selecting one of the lead stagger configurations comprises incrementally shifting one of the leads relative to another one of the leads in the graphical representation of the at least one lead stagger configuration.

13. The method of claim 10, wherein the function comprises conveying electrical energy from the actual leads to create a stimulation region within the tissue of the patient as the selected lead stagger configuration is graphically displayed.

14. The method of claim 13, wherein the function comprises moving the stimulation region relative to the actual leads as the selected lead stagger configuration is graphically displayed.

15. The method of claim 10, wherein the graphical representation of the selected lead stagger configuration includes electrodes.

16. The method of claim 15, wherein the function comprises displaying stimulation parameters adjacent the graphical representations of the electrodes.

17. The method of claim 16, wherein the displayed stimulation parameters include fractionalized electrical current values.

18. The method of claim 10, wherein the function comprises programming a control device configured controlling electrical stimulation energy provided to the actual electrode leads based on the selected lead stagger configuration.

19. A computer readable medium for programming a control device configured for controlling electrical stimulation energy provided to multiple electrode leads that are physically implanted within a patient in a side-by-side lead configuration, the computer readable medium containing instructions, which when executed, comprises:
displaying a graphical representation of at least one lead stagger configuration;
allowing a user to select one of the different lead stagger configurations by interacting with the displayed graphical representation of the at least one lead stagger configuration; and
performing a function with reference to the selected lead stagger configuration.

20. The computer readable medium of claim 19, wherein:
displaying the graphical representation of the at least one lead stagger configuration comprises simultaneously displaying a plurality of different lead stagger configurations and allowing includes allowing the user to select one of the displayed lead stagger configurations; or
allowing the user to select one of the lead stagger configurations comprises allowing the user to incrementally shift one of the leads relative to another one of the leads in the graphical representation of the at least one lead stagger configuration.
